(19) United States
(12) Patent Application Publication
(10) Pub. No.: US 2001/0041743 A1
(43) Pub. Date: Nov. 15, 2001

(54) Use of cyclopentenone derivatives for bone and periodontal regeneration

(76) Inventors: Steven Offenbacher, Chapel Hill, NC (US); Lyndon F. Cooper, Cary, NC (US); Michael E. Fritz, Atlanta, GA (US)

Correspondence Address: JENKINS & WILSON, PA, 3100 TOWER BLVD, SUITE 1400, DURHAM, NC, 27707, US

(21) Appl. No.: 09/760,080

(22) Filed: Jan. 12, 2001

Related U.S. Application Data
(60) Provisional application No. 60/175,813, filed on Jan. 12, 2000.

(51) Int. Cl. A61K 31/557

(52) U.S. Cl. 514/573; 424/423

(57) ABSTRACT

Methods for bone and periodontal tissue regeneration using cyclopentenone prostanoids and their agonists, and methods to retard pathophysiological calcification using cyclopentenone antagonists.

### CROSS REFERENCE TO RELATED APPLICATIONS

This application is based on and claims priority to U.S. Provisional Application Ser. No. 60/175,813, filed Jan. 12, 2000, herein incorporated by reference in its entirety.

### FIELD OF THE INVENTION

The present invention generally relates to regeneration of bone and periodontal tissue. Specifically, the present invention provides methods for bone and periodontal tissue regeneration using cyclopentenone prostanoids and their agonists, and methods to retard pathophysiological calcification using cyclopentenone antagonists.

### TABLE OF ABBREVIATIONS

| | |
|---|---|
| BMP-2 | bone morphogenetic protein 2 |
| BMP-6 | bone morphogenetic protein 6 |
| COX | cyclooxygenase enzyme complex |
| Δ¹²PGJ₂ | Δ¹²-13,14-dihydro-9-deoxy-Δ⁹- |
| | prostaglandin D₂, |
| EP₂ | a prostaglandin receptor |
| EP₄ | a prostaglandin receptor |
| IGF | insulin-like growth factor |
| M | molarity |
| MW | molecular weight |
| PDGF | platelet-derived growth factor |
| PDGF-A | platelet-derived growth factor A |
| | chain |
| PDGF-B | platelet-derived growth factor B |
| | chain |
| PG | prostaglandin |
| PGD₂ | prostaglandin D₂ |
| PGE₂ | prostaglandin E₂ |
| PGG₂ | prostaglandin G₂ |
| PGH₂ | prostaglandin H₂ |
| PGI₂ | prostaglandin I₂ |
| PGJ₂ | 9-hydroxy PGD₂ |

### BACKGROUND ART

Treatment of bone injury and pathophysiology are primary goals of modern periodontal and orthopedic therapy. Although bone serves as a structural support, it is a complex and metabolically active tissue that has the potential to resorb as well as to self-repair and to regenerate. Predictable regulation of these processes is the basis for development of new treatments for bone disorders. Further, periodontitis is a recognized risk factor for athersclerosis (Offenbacher et al. (1999) J Periodontal Res 34(7):346-352; Beck et al. (1999) Am Heart J 138(5 PT 2):S528-533). Thus, treatments that ameliorate periodontitis are also implicated as prophylactic measures for preventing or minimizing atherscierotic calcification.

Prostaglandins are potent stimulators of both bone formation and resorption. See Raisz (1999) Osteoarthritis and Cartilage 7:419-421; Jee and Ma (1997) Bone 21(4):297-304; Kawaguchi et al. (1996) Clin Orthopaed Rel Res 313:36-46; Bilezlkian et al. (eds) “Principles of Bone Biology” pp. 15-28 Academic Press, San Diego, Calif., USA. Most of the known stimulators of bone resorption activate COX-2, which in turn induces PG levels (Kawaguchi et al., 1996; Tai et al. (1997) Endocrinol 138:2372-2379; Min et al. (1998) J Bone Miner Res 13:1066-1075). Prostaglandins stimulate bone resorption by increasing the number and activity of osteoclasts (Woodiel (1996) J Bone Miner Res 11:1249-1255). PGE₂ is the most potent agonist, although other prostanoids, particularly PGI₂ are also potent stimulators. Prostaglandins can stimulate bone formation by promoting expression of growth factors that operate to increase replication and differentiation of osteoblasts.

The complex multifunctional regulation of PGs is thought to be mediated by different PG receptors. Two PG receptors have been identified in bone, the EP₂ and EP₄ receptors. The EP₄ receptor is implicated in the stimulation of bone resorption (Ono et al. (1998) J Endocrinol 158:R1-5). Bone formation is thought to be mediated by the EP₂ receptor, which is expressed in osteoblast precursor cells (Woodiel et al,1996; Nemoto (1997) Prostaglandins 54:713-725).

Skeletal tissue is an abundant source of PG production. Prostanoids are derived from membrane-associated arachidonic acid. Enzymes and factors regulating the metabolic conversion of arachidonic acid to Δ¹²PGJ₂ are thereby also regulators of bone growth. Arachidonate is a twenty-carbon fatty acid that is liberated from membrane phospholipids by the action of phospholipase A₂. In the presence of oxygen, arachidonic acid is rapidly converted to PGG₂, then further to PGH₂. These lipoxygenation reactions are mediated by a cyclooxygenase enzyme complex (COX). Endogenous PGs in bone are produced largely by induction of COX-2, which is principally regulated by hormones and local factors. Animals lacking COX-2 function by targeted knock-out of the COX-2 locus show impaired osteoclastogenesis (Raisz, 1999). In aqueous solution, PGH₂ rapidly decomposes into a mixture of PGE₂ and PGD₂. See Horner et al. (1996) Bone 19(4):353-362 and Hotz et al. (1994) Oral & Maxillofacial Surg 23:413-417. PGD₂ spontaneously degrades to 9-hydroxy PGD₂ (also known as PGJ₂) in aqueous solution, and is further converted to Δ¹²PGJ₂ in plasma (Jee et al. (1985) Calcified Tissue International 37(2):148-157; Klein-Nulend et al. (1997) Bone & Min Res 12(1):45-51).

The levels of PGE₂ and PGD₂ are differentially regulated in diverse cell types and in response to external stimuli by the collective activity of several enzymes, including PGD₂ synthase and PGD₂ isomerase. Synthesis of PGD₂ from PGH₂ is mediated by PGD₂ synthase. Alternatively, PGD₂ can be derived from PGE₂ by the action of a PGD₂ isomerase. PGD₂ is produced in a wide variety of cells including macrophages, platelets, and mast cells, and it is the major arachidonic acid metabolite produced by osteoblasts isolated from chick calvaria and bone marrow (Hotz et al., 1994; Howell et al. (1997) J Periodontol 68(12):1186-1193; Hughes et al. (1992) Bone & Mineral 19(1):63-74; Jee (1992) Bone 13(2):153-159).

The goal of bone regenerative procedures isto induce osseus progenitor cells to replicate and differentiate into new supporting tissues, including new supporting bone, cementum, and periodontal ligament. Thus, substances affecting the recruitment, proliferation, and differentiation of osteoprogenitor cells are potential therapeutic compounds for bone regeneration (Dover et al. (1994) Histochemistry 102(5):383-387; Feyen et al. (1984) Prostaglandins 28(6):769-778). Conversely, therapeutic compositions for treatment of periodontitis and other diseases of bone growth inhibit this process.

As disclosed herein, cyclopentenone compounds, cyclopentenone analogs, and substances that regulate cyclopentenone synthesis are useful for promoting bone regeneration, bone repair, bone growth, periodontal tissue regeneration, periodontal tissue repair, and periodontal tissue growth. Also disclosed are methods for using cyclopentenone compound antagonists and substances that disrupt cyclopentenone synthesis for treatment of calcification associated with athersclerotic diseases. By provision of therapeutic compositions comprising cyclopentenone compounds, therapeutic compositions comprising cyclopentenone modulator compounds, and methods for using the same, the present invention meets a long-felt need for improved therapies for bone injury and disease.

### SUMMARY OF THE INVENTION

The present invention discloses therapeutic compounds and compositions for bone growth and repair comprising cyclopentenone compounds and cyclopentenone related compounds. Preferably, the cyclopentenone compound or cyclopentenone related compound is of the formula 2-R¹, 1-R², cyclopent 3-en 1-one or 6274-hydroxy, 3-R¹, 2-R², cyclopentan-1-one, wherein R¹ and R² are each independently aliphatic chains. In one embodiment, R¹ and R² are each independently substituted or unsubstituted C₁-C₁₀ straight chain or branched alkyl, or substituted or unsubstituted C₂-C₁₀ straight chain or branched alkenyl, substituted or unsubstituted C₂-C₁₀ straight chain or branched alkynyl; and wherein the substituents are each independently halogen, cyano, amino, carboxy, ester, ether, carboxamide, hydroxy, or mercapto. The disclosed cyclopentenone related compounds comprise compounds that induce or enhance cyclopentenone synthesis by a cell or compounds that are cyclopentenone agonists.

The present invention also discloses methods for identifying cyclopentenone modulators. According to the method, a cyclopentenone or cyclopentenone related compound is exposed to a plurality of candidate modulators. A cyclopentenone modulator is selected as a substance that specifically binds the target cyclopentenone or cyclopentenone related compound. A cyclopentenone modulator can be a protein, peptide, antibody, chemical compound, or nucleic acid.

Preferred therapeutic compositions comprising cyclopentenone compounds, cyclopentenone related compounds, or cyclopentenone modulators include a pharmaceutically acceptable carrier such as a membrane, a film, a matrix, a scaffold, an implantable device, a biodegradable carrier, a slow release carrier, a controlled release carrier, a liposome, or a microparticle. In one embodiment, the implantable device is a collagen device. In an alternative embodiment, the implantable device is a titanium support.

The present invention also provides a method of stimulating bone growth. According to the method, a composition is prepared comprising one or more of the disclosed cyclopentenone compounds, cyclopentenone related compounds, or cyclopentenone modulators; and a carrier. A composition thus prepared is administered to a subject, whereby bone growth is stimulated. The method can employ naturally occurring cyclopentenone compounds, preferably Δ¹²-13,14-dihydro-9-deoxy-Δ⁹-prostaglandin D₂ 9-hydroxy-prostaglandin D₂, prostaglandin D₂, derivatives thereof, salt forms thereof, and combinations thereof. Alternatively, synthetic, non-naturally occurring compounds can be used. Preferably, the disclosed cyclopentenone composition is administered to the subject at a site of bone injury or disease. In one embodiment, the site of bone injury or disease is an intraoral site, and the method can further comprise implantation of a tooth or tooth implant. Preferably, bone growth or repair conferred by administration of the disclosed cyclopentenone compounds comprises an increase in bone volume. Bone growth or repair according to the disclosed method also preferably comprises elevated expression of PDGF, BMP-2, BMP-6, and combinations thereof.

Accordingly, it is an object of the present invention to provide novel compositions and methods to promote bone regeneration, bone repair, bone growth, periodontal tissue regeneration, periodontal tissue repair, and periodontal tissue growth.

An object of the invention having been stated above, other objects and advantages of the present invention will become apparent to those skilled in the art after a study of the following description of the invention, Figures and non-limiting Examples.

### BRIEF DESCRIPTION OF THE DRAWINGS

FIG. 1 depicts an exemplary titanium enclosure model as described in Example 1.

FIG. 2 graphically illustrates an increase in bone volume following Δ¹²PGJ₂ administration.

FIG. 3 is a diagram of an implant indicating sites for bone height (1) and bone width (the mean of 2, 3, and 4) measurements as descriptors of bone growth. Significantly increased bone growth is observed at 3 weeks and 8 weeks following administration of Δ¹²PGJ₂ compared to administration of a control vehicle.

FIG. 4 graphically illustrates an increase in PDGF-B expression following administration of Δ¹²PGJ₂.

FIG. 5 graphically illustrates an increase in BMP-6 expression following administration of Δ¹²PGJ₂.

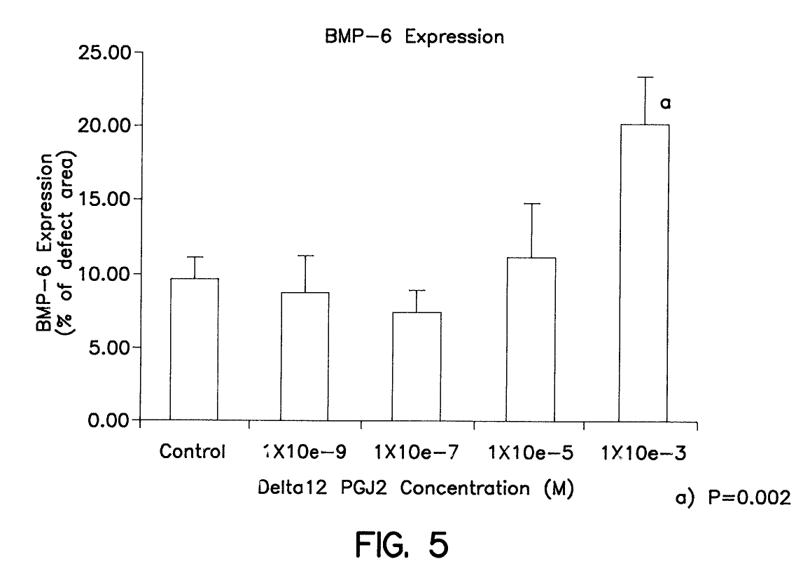

### DETAILED DESCRIPTION OF THE INVENTION

The present invention discloses methods for using cyclopentenone compounds, cyclopentenone analogs, and substances that regulate cyclopentenone synthesis for promoting bone regeneration, bone repair, bone growth, periodontal tissue regeneration, periodontal tissue repair, and periodontal tissue growth. Also disclosed are methods for using cyclopentenone compounds, cyclopentenone analogs, and substances that regulate cyclopentenone synthesis for treatment of calcification associated with athersclerotic diseases.

The disclosed cyclopentenone compounds and cyclopentenone related compounds can be combined with other biologically active molecules as well as carriers, carrier agents, vehicles, and other drug delivery compositions. Several other types of compounds can be used that can produce similar effects. These include molecules that induce or enhance cyclopentenone synthesis, such as COX-1, activators of COX-2, or analogues of arachidonic acid, can have, by virtue of the production of natural cyclopentenones, similar effects as the disclosed cyclopentenone compounds; molecules that act as cyclopentenone agonists to induce production of PDGF and BMP and bone formation; and compounds that target the nuclear PPAR transcriptional factor involved in activation of wound healing or repair.

As used herein, the term cyclopentenone compound refers to actual cyclopentenones such as Δ¹² PGJ₂, PGJ₂, and PGD₂ and their various forms. The term cyclopentenone related compounds refers to compounds that enhance synthesis of cyclopentenone by cells, compounds that act as cyclopentenone agonists to induce PDGF, BMP, and bone formation, and compounds that target the nuclear PPAR transcriptional factor involved in activation of wound healing or repair. Compounds that target the nuclear PPAR transcriptional factor involved in activation of wound healing or repair would, as do the disclosed cyclopentenone compounds, form an activating complex with the nuclear PPAR transcriptional factor which in turn would activate wound healing and repair genes. Thus, all of the disclosed cyclopentenone related compounds will produce effects similar to the disclosed cyclopentenone compounds by causing an increase in cyclopentenones in cells, causing induction of bone formation factors, or activating genes that are activated by cyclopentenones. The usefulness of all of these compounds comes from the discovery of the effect of cyclopentenone compounds on bone formation and the bone formation pathway.

### I. Δ¹²PGJ₂ Activity in Promoting Bone Growth

### I. A. Cyclopentenone Compounds and Compositions

Cyclopentenone compounds useful in the disclosed methods include Δ¹²-13,14-dihydro-9-deoxy-A9-prostaglandin D₂ (Δ¹²PGJ₂), 9-hydroxy prostaglandin D₂ (PGJ₂), and prostaglandin D₂ (PGD₂). Multiple forms of the disclosed cyclopentenone compounds can also be used as disclosed. These forms include various derivatives, such as methyl-ester derivatives, isoforms, salt forms, and cyclopentenone compounds structurally related to Δ¹²PGJ₂, PGJ₂, and PGD₂.

A general formula for PG cyclopentenone compounds is 2-R¹, 1-R², cyclopent 3-en 1-one, where R¹ and R² are each independently aliphatic chains with or without unsaturation or substitution. R¹ and R² are preferably substituted or unsubstituted C₁-C₁₀ straight chain or branched alkyl, substituted or unsubstituted C₂-C₁₀ straight chain or branched alkenyl, or substituted or unsubstituted C₂-C₁₀ straight chain or branched alkynyl, where the substituents are each independently halogen, cyano, amino, carboxy, ester, ether, carboxamide, hydroxy, or mercapto.

A general formula for PGD cyclopentenone compounds is 4-hydroxy, 3-R¹, 2-R², cyclopentan-1-one, where R¹ and R² are each independently aliphatic chains with or without unsaturation or substitution. R¹ and R² are preferably substituted or unsubstituted C₁-C₁₀ straight chain or branched alkyl, substituted or unsubstituted C₂-C₁₀ straight chain or branched alkenyl, or substituted or unsubstituted C₂-C₁₀ straight chain or branched alkynyl, where the substituents are each independently halogen, cyano, amino, carboxy, ester, ether, carboxamide, hydroxy, or mercapto.

The term “cyclopentenone related compounds” refers to compounds that enhance synthesis of cyclopentenone by cells and to compounds that act as cyclopentenone agonists. The term “cyclopentenone related compounds” includes compounds that induce PDGF and BMP expression and promote bone formation.

The term “cyclopentenone composition”, as used herein, refers to a mixture, solution, or other combination comprising the disclosed cyclopentenone and cyclopentenone related compounds. For example, the disclosed cyclopentenone and cyclopentenone related compounds can be combined with other biologically active molecules as well as carriers, scaffolds, matrices, implants, devices, and any other delivery vehicle. The disclosed cyclopentenone and cyclopentenone related compounds can also be combined with other therapeutic agents, including other compounds that promote tissue growth or infiltration, such as growth factors. Exemplary growth factors for this purpose include epidermal growth factor, fibroblast growth factor, platelet-derived growth factor, transforming growth factors, parathyroid hormone, leukemia inhibitory factor, and insulin-like growth factors. Agents that promote bone growth such as bone morphogenetic protein (U.S. Pat. No.4,761,471; PCT International Publication WO 90/11366), osteogenin (Sampath et al. (1987) Proc Natl Acad Sci USA 84:109-113), and NaF (Tencer et al. (1989) J Biomed Mat Res 23:571-589) can also be used. Other biologically active agents include enzymes, antibodies, chemotherapeutic agents, insulin, or enzyme inhibitors. The disclosed compounds can also be used in conjunction with agents that inhibit bone resorption. Antiresorptive agents for this purpose include but are not limited to estrogen, bisphosphates, and calcitonin.

### I. B. In vivo Models of Bone Growth

The disclosed compounds can be used to stimulate growth of bone-forming cells or their precursors, or to induce differentiation of bone-forming cell precursors, either in vitro or ex vivo. As used herein, the term “precursor cell” refers to a cell that is committed to a differentiation pathway, but that generally does not express markers or function as a mature, fully differentiated cell. As used herein, the term “mesenchymal cells” or “mesenchymal stem cells” refers to pluripotent progenitor cells that are capable of dividing many times, and whose progeny will give rise to skeletal tissues, including cartilage, bone, tendon, ligament, marrow stroma and connective tissue. See Caplan (1991) J Orthop Res 9:641-650. As used herein, the term “osteogenic cells” includes osteoblasts and osteoblast precursor cells.

Two different animal model systems were used to demonstrate the effects of administering Δ¹²PGJ₂ on bone regeneration in vivo, as described in Examples 1 and 2.

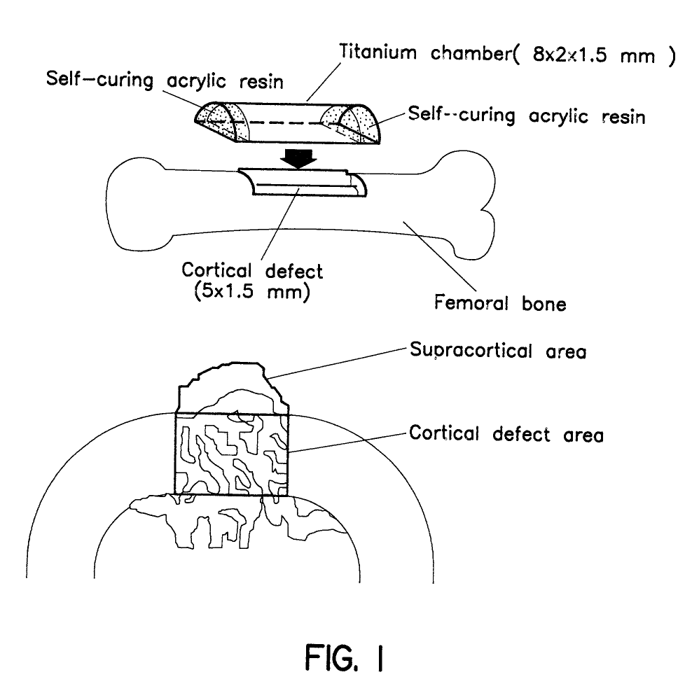

In one study, bone regeneration and gene expression was assayed using a titanium enclosure model, as described in Example 1 (FIG. 1). Briefly, transcortical defects were created surgically in rat femurs. A collagen sponge, loaded with a varying concentration of Δ¹²PGJ₂ was applied to each defect. The collagen sponge was secured by placement of the titanium enclosure, which served as a rigid and fixed geometry cell exclusion barrier. Animals were sacrificed at 10 days and bone tissue sections were prepared for immunohistology to quantify growth factor expression. Tissue sections from treated animals were also analyzed morphometrically to assess de novo bone formation of the defect areas.

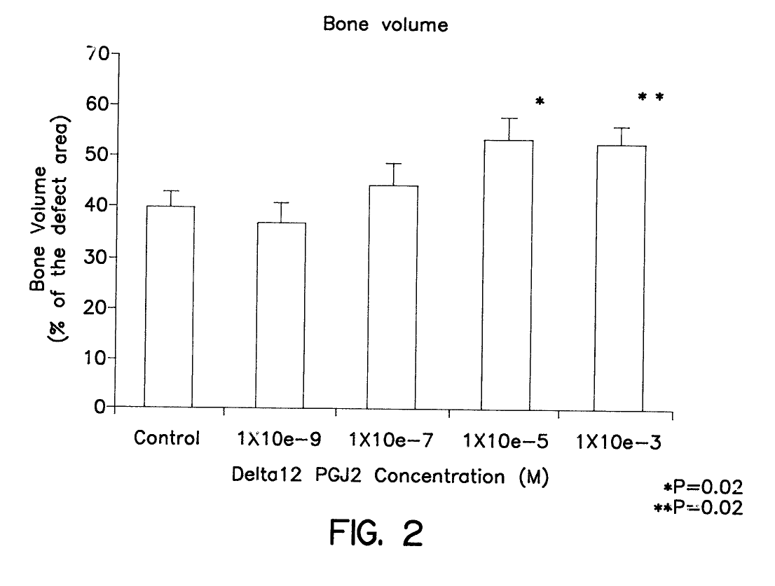

Post-operative analysis supported a role for Δ¹²PGJ₂ in promoting bone growth. Histological examination of control enclosures revealed that the enclosure space contained granulation tissue with small blood vessels. Some collagen carrier and a partly disintegrated blood clot were also present at the upper part of the enclosure space. The cortical defect area in all samples was partially filled with newly formed trabecular bone containing some connective tissue and bone marrow spaces. The surface of all remodeling trabecular bone showed areas of active osteoblastic bone formation as well as osteoclastic resorption. New bone formation activity was observed as numerous osteoblasts on trabecular bone adjacent to granulation tissue in the enclosure space. The newly formed bone trabeculae advanced from sides of the surgically cut cortical bone as well as from the bone marrow and endosteal surfaces and tended to be only weakly stained by eosin. Histomorphometic analysis of the cortical defect area indicated that the percentage of new bone formation increased in a dose-dependent manner following administration of Δ¹²PGJ₂. 3.0 μg and 30 ng doses of Δ¹²PGJ₂ induced significant increases in bone growth (54% and 53% volume, respectively) compared to a control value (40%) (FIG. 2). This represented approximately a 33% increase in total bone area following Δ¹²PGJ₂ treatments.

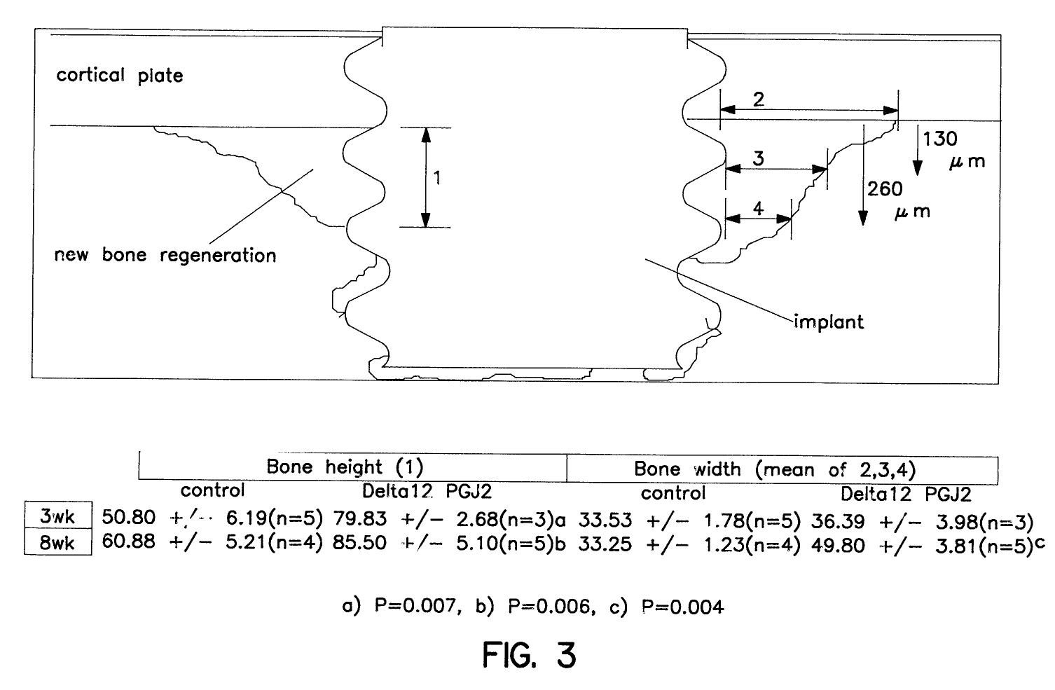

In a second study, titanium implants were placed in the rat femurto mimic human dental implant placement, as described in Example 2. Bone healing and regeneration around the implants was assessed following treatment with implants loaded with Δ¹²PGJ₂. Bone height and width were significantly increased in Δ¹²PGJ₂-treated injuries compared to control injuries (FIG. 3). Thus, cyclopentenone administration resulted in a more rapid bone apposition and integration to the implant surface, and also promoted a thicker bone configuration around the implant.

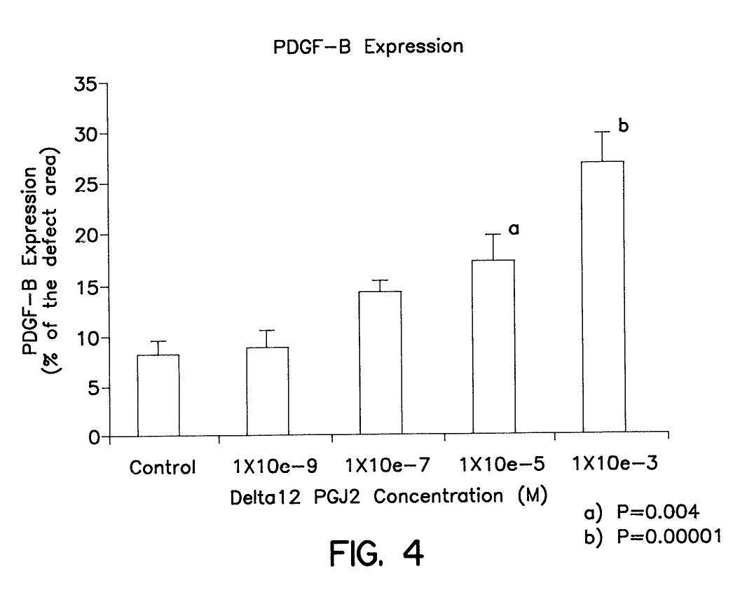

PDGF expression was observed in osteoblast cells, in osteocytes in bone trabeculae, and in cells within the bone marrow space (e.g. megakaryocytes). A significant increase of PDGF-B expression in the area of the defect was observed following treatment with 10⁻³M or 10⁻⁵M Δ¹²PGJ₂ (FIG. 4). A dose response relationship between PDGF expression and the concentration of Δ¹²PGJ₂ was observed. PDGF was predominantly detected in osteocytes and osteoblasts, and was also sporadically present in stromal fibroblasts in the connective tissue adjacent to bone trabeculae.

Summarily, these studies demonstrate that Δ¹²PGJ₂ promotes bone regeneration in a dose-dependent manner. A second key observation was that this cyclopentenone is a potent and dose-dependent inducer of several key anabolic bone growth and differentiation factors, including PGDF-A and -B, BMP-2 and BMP-6. These molecules are induced following Δ¹²PGJ₂ administration and act in concert to result in an increase in bone volume, an increase in bone trabecular density, and earlier formation of mature bone. The faster bone regeneration that occurred as a result of Δ¹²PGJ₂ administration hastens functional loading of the injured bone.

The present invention discloses that the pathway of PGD₂→PGJ₂→Δ¹²PGJ₂→→PDGF, BMP-2 and BMP-6 is a critical regulator event in triggering bone regeneration. Biodegradable matrices containing PGD₂, PGJ₂, Δ¹²PGJ₂, or functional analogues thereof are capable of inducing bone regeneration and improving bone repair. This activity is achieved in part through induction of mesenchymal growth factors including PDGF, BMP-6 and BMP-2.

### II. Identification of Cyclopentenone Modulators

The present invention further discloses a method for identifying a compound that modulates cyclopentenone function in vivo, such as Δ¹²PGJ₂ function. According to the method, a cyclopentenone is exposed to a plurality of compounds, and binding of a compound to the isolated cyclopentenone is assayed. A compound is selected that demonstrates specific binding to the cyclopentenone.

The term “cyclopentenone modulator”, as used herein, refers to any substance that mimics, promotes, disrupts, or otherwise regulates the activity of an endogenous cyclopentenone or a cyclopentenone that has been administered to a subject. The term “regulates” refers to both upregulation and abrogation of cyclopentenone activity. A cyclopentenone modulator can regulate a cyclopentenone by direct binding or physical interaction with the cyclopentenone. Alternatively, a cyclopentenone modulator can function indirectly, for example via interaction or binding with a factor that impacts cyclopentenone synthesis or stability. “Cyclopentenone modulators” include cyclopentenone related compounds as defined herein above.

Candidate regulators include but are not limited to proteins, peptides, antibodies, chemical compounds, and nucleic acids. Structural analysis of these selectants can provide information about cyclopentenone/modulator interactions that enable the development of pharmaceuticals based on these lead structures.

Similarly, the knowledge of the structure of a native cyclopentenone (e.g., Δ²PGJ₂) provides an approach for rational drug design. See Huang et al. (2000) Pac Symp Biocomput 230-41; Saqi et al. (1999) Bioinformatics 15:521-522. Computer models can further predict binding of a native cyclopentenone (e.g., Δ¹²PGJ₂) or cyclopentenone related compounds to various candidate modulators that can be synthesized and tested. Additional drug design techniques are described in U.S. Pat. Nos. 5,834,228 and 5,872,011.

The term “binding” and “interaction” each refer to an affinity between two molecules, for example, a ligand and a receptor. As used herein, “binding” means a preferential binding of one molecule for another in a mixture of molecules. The binding of the molecules can be considered specific if the binding affinity is about 1×10⁴ M⁻¹ to about 1×10⁶ M⁻¹ or greater. Binding of two molecules also encompasses a quality or state of mutual action such that an activity of one molecule on another molecule is inhibitory (in the case of an antagonist) or enhancing (in the case of an agonist). Exemplary binding assays include but are not limited to Fluorescence Correlation Spectroscopy (FCS), Surface-Enhanced Laser Desorption/Ionization time-of-flight mass spectrometry (SELDI-TOF), and Biacore, each described further herein below.

Fluorescence Correlation Spectroscopy (FCS) measures the average diffusion rate of a fluorescent molecule within a small sample volume (Madge et al. (1972) Phys Rev Lett 29:705-708; Maiti et al. (1997) Proc Natl Acad Sci USA, 94:11753-11757). The sample size can be as low as 10³ fluorescent molecules and the sample volume as low as the cytoplasm of a single bacterium. The diffusion rate is a function of the mass of the molecule and decreases as the mass increases. FCS can therefore be applied to protein-ligand interaction analysis by measuring the change in mass and therefore in diffusion rate of a molecule upon binding. In a typical experiment, the target molecule is labeled with a fluorescent tag. The target molecule is then exposed in solution to the potential ligand, and its diffusion rate is determined by FCS using instrumentation available from a variety of sources, including Carl Zeiss, Inc. of Thornwood, N.Y. Ligand binding is determined by changes in the diffusion rate of the labeled target molecule.

Surface-Enhanced Laser Desorption/Ionization (SELDI) was developed by Hutchens and Yip (1993) Rapid Commun Mass Spectrom 7:576-580). When coupled to a time-of-flight mass spectrometer (TOF), SELDI provides a means to rapidly analyze molecules retained on a chip. It can be used to analyze molecular interactions, for example, by covalently binding the target molecule on the chip and assaying by mass sepctrometry (MS) small molecules that bind to a chip (Worrall et al. (1998) Anal Biochem 70:750-756). The chip thus prepared is then exposed to the potential ligand via, for example, a delivery system able to pipet candidate substances in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Substances that specifically bind the target are identified by the stringency of the wash needed to elute them.

Biacore relies on changes in the refractive index at the surface layer upon binding of a candidate substance to a target molecule immobilized on the layer. In this system, a collection of small candidate substances is injected sequentially in a 2-5 microliter cell, wherein the target molecule is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein (Liedberg et al. (1983) Sensors Actuators 4:299-304; Malmquist (1993) Nature 361:186-187). The SPR signal on the chip is recorded and changes in the refractive index indicate an interaction between the immobilized target and the candidate modulator. Analysis of the signal kinetics of on rate and off rate allows the discrimination between non-specific and specific interaction.

### III. Therapeutic Methods

The present invention further provides methods for employing the disclosed cyclopentenone compounds, cyclopentenone related compounds, and cyclopentenone modulators as pharmaceutical compositions. The term “pharmaceutical composition” or “drug” as used herein, each refer to any substance having a biological activity. Substances discovered by methods of the present invention include but are not limited to proteins, peptides, chemical compounds, antibodies, and nucleic acids.

### III. A. Subjects

With respect to the therapeutic methods of the present invention, a preferred subject is a vertebrate subject. A preferred vertebrate is warm-blooded; a preferred warm-blooded vertebrate is a mammal. A preferred mammal is a mouse or, most preferably, a human. As used herein and in the claims, the term “patient” includes both human and animal patients. Thus, veterinary therapeutic uses are provided in accordance with the present invention.

Also provided is the treatment of mammals such as humans, as well as those mammals of importance due to being endangered, such as Siberian tigers; of economical importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, provided is the treatment of livestock, including, but not limited to, domesticated swine, ruminants, ungulates, horses, poultry, and the like.

As used herein, the term “experimental subject” refers to any subject or sample in which the desired measurement is unknown. The term “control subject” refers to any subject or sample in which a desired measure is unknown.

### III. B. Formulations and Delivery Devices

A composition of the present invention is typically formulated using acceptable vehicles, adjuvants, and carriers as desired.

Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectable compositions.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic diluent or solvent, for example 1,3-butanediol.

The disclosed compounds can be incorporated into or used with a variety of implants and other devices. Many such implants, also referred to herein as “implantable devices”, are known, including but not limited to titanium supports, screws, bolts, wires, plates, bars, sponges, meshes, space-filling matrices, and shape-forming matrices.

Matrix. A preferred material for use with the disclosed cyclopentenone compounds and cyclopentenone related compounds is matrix. Matrix material includes bone-derived material; organic or inorganic material such as hydroxyapatite and tricalcium phosphate (TCP); natural or synthetic polymeric materials including biodegradable polymers such as polyhydroxy acids, polyanhydrides, polyesters, polylactic acid, polyglycolic acid, polybutyric acid, and non-degradable polymers such as polycarbonate, polyacrylates, and ethylenevinylacetate.

The surface charge, particle size, the presence of mineral, and the methodology for combining matrix and the disclosed compounds can play a role in achieving successful bone induction. Perturbation of the charge by chemical modification can abolish the inductive response. Particle size can influence the quantitative response of new bone; particles between 70 μm and 420 μm are preferred as they appear to elicit the maximum response. Contamination of the matrix with bone mineral can inhibit bone formation.

The sequential cellular reactions in the interface of the bone matrix/compound implants are complex. The multistep cascade includes: binding of fibrin and fibronectin to implanted matrix, migration and proliferation of mesenchymal cells, differentiation of the progenitor cells into chondroblasts, cartilage formation, cartilage calcification, vascular invasion, bone formation, remodeling, and bone marrow differentiation.

A successful matrix for the disclosed compounds preferably performs several important functions. It should carry the compound and act as a slow release delivery system, and accommodate each step of the cellular response during bone development. In addition, selected materials should be biocompatible in vivo and preferably biodegradable; the matrix should act as a temporary scaffold until replaced completely by new bone. Polylactic acid (PLA), polyglycolic acid (PGA), and various combinations have different dissolution rates in vivo. In bones, the dissolution rates can vary according to whether the implant is placed in cortical or trabecular bone. Matrix geometry, particle size, the presence of surface charge, and the degree of both intra-and inter-particle porosity are all important to successful matrix performance. It is preferred to shape the matrix to the desired form of the new bone and to have dimensions that span non-union defects. The new bone formed with matrix has essentially the dimensions of the device implanted. The matrix can comprise a shape-retaining solid made of loosely adhered particulate material, for example, collagen. It can also comprise a molded, porous solid, or simply an aggregation of close-packed particles held in place by surrounding tissue. Masticated muscle or other tissue can also be used. Large allogenic bone implants can act as a substrate for the matrix if their marrow cavities are cleaned and packed with particles and the dispersed compound.

A preferred matrix material, prepared from xenogenic bone and treated as disclosed herein, produces an implantable material useful in a variety of clinical settings. In addition to its use as a matrix for bone formation in various orthopedic, periodontal, and reconstructive procedures, the matrix also can be used as a sustained release carrier, or as a collagenous coating for implants. The matrix can be shaped as desired in anticipation of surgery or shaped by the physician or technician during surgery. Thus, the material can be used for topical, subcutaneous, intraperitoneal, or intramuscular implants; it can be shaped to span a nonunion fracture or to fill a bone defect. In bone formation procedures, the material is slowly absorbed by the body and is replaced by bone in the shape of or very nearly the shape of the implant. Those skilled in the art can create a biocompatible matrix of choice, for use with the disclosed compounds, having a desired porosity or surface microtexture useful in the production of osteogenic devices, and useful in other implantable contexts, for example, as a packing to promote bone induction, or as a biodegradable sustained release implant. In addition, synthetically formulated matrices, prepared as disclosed herein, can be used.

Demineralized bone matrix, preferably bovine bone matrix can be prepared as described in Example 3. Bone matrix so prepared can be further treated to remove pathogens prior to implantation by treatment with acids or solvents as described in Example 3. The matrix can also be treated to remove contaminating heavy metals, also described in Example 3.

The collagen materials of bone matrix preferably take the form of a fine powder, insoluble in water, comprising nonadherent particles. The matrix, in combination with the disclosed compounds, can be used by packing into the volume where new bone growth or sustained compound release is desired. The matrix is held in place by surrounding tissue. Alternatively, the powder can be encapsulated in, for example, a gelatin or polylactic acid coating, which is absorbed readily by the body. The powder can be shaped to a volume of given dimensions and held in that shape by interadhering the particles using, for example, soluble, species-biocompatible collagen. The material can also be produced in a sheet, rod, bead, or other macroscopic shapes.

Useful matrices can also be formulated synthetically. One example of such a synthetic matrix is the porous, biocompatible, in vivo biodegradable synthetic matrix disclosed in U.S. Pat. No. 5,645,591. Briefly, the matrix comprises a porous crosslinked structural polymer of biocompatible, biodegradable collagen, most preferably tissue-specific collagen, and appropriate, tissue-specific glycosaminoglycans as tissue-specific cell attachment factors. Bone tissue-specific collagen (e.g., Type I collagen) derived from a number of sources can be suitable for use in these synthetic matrices, including soluble collagen, acid-soluble collagen, collagen soluble in neutral or basic aqueous solutions, as well as those collagens which are commercially available. In addition, Type II collagen, as found in cartilage, also can be used in combination with Type I collagen.

Glycosaminoglycans (GAGs) or mucopolysaccharides are polysaccharides made up of residues of hexoamines glycosidically bound and alternating in a more-or-less regular manner with either hexouronic acid or hexose moieties. GAGs are of animal origin and have a tissue specific distribution. Reaction with the GAGs also provides collagen with another valuable property, i.e., inability to provoke an immune reaction (foreign body reaction) from an animal host.

Useful GAGs include those containing sulfate groups, such as hyaluronic acid, heparin, heparin sulfate, chondroitin 6-sulfate, chondroitin 4-sulfate dermatan sulfate, and keratin sulfate. For osteogenic devices chondroitin 6-sulfate is preferred. Other GAGs also can be suitable for forming the matrix described herein, and those skilled in the art will either know or be able to ascertain other suitable GAGs using no more than routine experimentation after a review of the disclosure of the present invention presented herein. For a more detailed description of mucopolysaccharides. See Aspinall (1970) “Polysaccharides” Pergamon Press, Oxford, United Kingdom.

Collagen can be reacted with a GAG in aqueous acidic solutions, preferably in diluted acetic acid solutions. By adding the GAG dropwise into the aqueous collagen dispersion, coprecipitates of tangled collagen fibrils coated with GAG result. This tangled mass of fibers then can be homogenized to form a homogeneous dispersion of fine fibers and then filtered and dried.

Insolubility of the collagen-GAG products can be raised to the desired degree by covalently cross-linking these materials, which also serves to raise the resistance to resorption of these materials. In general, any covalent cross-inking method suitable for cross-linking collagen also is suitable for cross-inking these composite materials, although cross-linking by a dehydrothermal process is preferred.

Another useful synthetic matrix is one formulated from biocompatible, in vivo biodegradable synthetic polymers, such as those composed of glycolic acid, lactic acid and/or butyric acid, including copolymers and derivatives thereof. These polymers are well described in the art and are available commercially. For example, polymers, composed of polylactic acid (e.g., MW 100 kDa), 80% polylactide/20% glycoside or poly 3-hydroxybutyric acid (e.g., MW 30 kDa) can be purchased from PolySciences, Inc. of Warrington, Pa. The polymer compositions are generally obtained in particulate form and the osteogenic devices preferably fabricate under nonaqueous conditions (e.g., in an ethanol-trifluoroacetic acid solution, EtOH/TFA) to avoid hydrolysis of the polymers. In addition, one can alter the morphology of the particulate polymer compositions, for example to increase porosity, using any of a number of particular solvent treatments known in the art.

The disclosed compounds can be combined and dispersed in a suitable bone derived or synthetic matrix as described in Example 4. Additional therapeutic drugs, hormones, and various bioactive species can be included in a cyclopentenone matrix composition using similar methods.

Liposomes. The disclosed compounds can also be incorporated into liposomes by any of the reported methods of preparing liposomes. The present compositions can utilize the compounds noted above incorporated into liposomes in order to direct these compounds to cells and tissues and organs which take up the liposomal composition. The liposome-incorporated compounds can be utilized by implantation or by parenteral administration, to allow for the efficacious use of lower doses of the compounds. Ligands can also be incorporated to further focus the specificity of the liposomes.

Suitable conventional methods of liposome preparation include, but are not limited to, those disclosed by Bangham et al. (1965) J Mol Biol 23:238-252; Olson et al. (1979) Biochim Biophys Acta 557:9-23; Szoka et al. (1978) Proc Natl Acad Sci USA 75:4194-4198; Kim et al. (1983) Biochim Biophys Acta 728:339-348; and Caner et al. (1986) Biochim Biophys Acta 858:161-168.

The liposomes can be made from the present compounds in combination with any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine, or phosphatidylinositol. Synthetic phospholipids that can also be used, include, but are not limited to: dimyristoylphosphatidylcholine, dioleoylphosphatidylcholine, dipalmitoylphosphatidylcholine, distearoylphosphatidycholine, and the corresponding synthetic phosphatidylethanolamines and phosphatidylglycerols. Cholesterol or other sterols, cholesterol hemisuccinate, glycolipids, cerebrosides, fatty acids, gangliosides, sphingolipids, 1,2-bis(oleoyloxy)-3-(trimethyl ammonio) propane (DOTAP), N-1-(2,3-dioleoyl) propyl-N,N,N-trimethylammonium chloride (DOTMA), and other cationic lipids can be incorporated into the liposomes by methods known to those skilled in the art. The relative amounts of phospholipid and additives used in the liposomes can be varied if desired. The preferred ranges are from about 60 to 90 mole percent of the phospholipid; cholesterol. Cholesterol hemisuccinate, fatty acids or cationic lipids can be used in amounts ranging from 0 to 50 mole percent. The amounts of the present compounds incorporated into the lipid layer of liposomes can be varied with the concentration of their lipids ranging from about 0.01 to about 50 mole percent.

The liposomes with the above formulations can be made still more specific for their intended targets with the incorporation of monoclonal antibodies or other ligands specific for a target. For example, monoclonal antibodies to the BMP receptor can be incorporated into the liposome by linkage to phosphatidylethanolamine (PE) incorporated into the liposome by the method of Lesernian et al. (1980) Nature 288:602-604.

Microparticles. The disclosed compounds can also be incorporated into microparticles for administration. The term “microparticles” encompasses microcapsules, microspheres and nanoparticles, with the understanding that the actual conformation of the particles will be determined by the chemical composition of the particle and method of manufacture. Preferably, microspheres and microcapsules (having a core of a different material than the outerwall), have a diameter from nanometers to 5000 microns. Microparticles of less then ten microns and more preferably less than five microns are preferred for uptake by phagocyte cells.

The microparticles can consist entirely of polymer or have only an outer coating of polymer. Microparticles can also consist of non-polymeric materials, such as liposomes. Exemplary methods for constructing and loading microparticles include single and double emulsion solvent evaporation, spray drying, solvent extraction, solvent evaporation, phase separation, simple and complex coacervation, and interfacial polymerization, as described in Example 5. Additional methods for making drug delivery microparticles can be found in, for example, Doubrow (ed) (1992) “Microcapsules and Nanoparticies in Medicine and Pharmacy” CRC Press, Boca Raton and Benita (ed) (1996) “Microencapsulation: Methods and Industrial Applications” Marcel Dekker, Inc., New York.

Carriers and Excipients. Aqueous suspensions can contain the disclosed compounds in admixture with pharmacologically acceptable excipients, comprising suspending agents, such as methyl cellulose, and wetting agents, such as lecithin, lysolecithin, and long-chain fatty alcohols. The aqueous suspensions can also contain preservative, coloring agents, flavoring agents, and sweetening agents in accordance with industry standards.

Preparations for topical and local application can comprise aerosol sprays, lotions, gels, and ointments in pharmaceutically appropriate vehicles which ay comprise lower aliphatic alcohols, polyglycols such as glycerol, polyethylene glycol, esters of fatty acids, oils, fats, and silicones. The preparations can further comprise antioxidants, such as ascorbic acid or tocopherol, and preservatives, such a p-hydroxybenzoic acid esters.

Parenteral preparations typically comprise particularly sterile or sterilized products. Injectable compositions can be provided containing the disclosed compound and any well-known injectable carrier. These can also contain salt for regulating osmotic pressure.

### III. C. Delivery Methods

For the purposes described herein, the identified substances can normally be administered systemically, parenterally, or orally. The term “parenteral” as used herein includes intravenous, intra-muscular, intra-arterial injection, or infusion techniques. The identified substances can also be delivered by implantation of a pharmaceutically acceptable carrier, as disclosed herein, at a site of interest, wherein the delivery vehicle comprises the identified substances.

### III. D. Administration Profiles

The disclosed cyclopentenone and cyclopentenone related compounds can be formulated, used in combination with drug delivery devices, and administered so as to confer controlled drug release. Exemplary drug release profiles include continuous or sustained release, variable release, and intermittent release. Intravenous administration will generally comprise a series of injections or continuous infusion over an extended temporal period. Administration of the disclosed compounds by injection will generally occur at discretely spaced weekly or daily intervals. Alternatively, the compounds disclose herein can be administered in a pulsatile manner. In all cases, treatments are intended to continue until the desired outcome is achieved.

### III. E. Dose

As used herein, an “effective” dose refers to a dose(s) administered to an individual patient sufficient to cause a change in activity of cyclopentenone and cyclopentenone related compounds. One of ordinary skill in the art can tailor the dosages to an individual patient, taking into account the particular formulation and method of administration to be used with the composition as well as patient height, weight, severity of symptoms, and stage of the biological condition to be treated. Such adjustments or variations, as well as evaluation of when and how to make such adjustments or variations, are well known to those of ordinary skill in the art of medicine.

A therapeutically effective amount can comprise a range of amounts. One skilled in the art can readily assess the potency and efficacy of a cyclopentenone or cyclopentenone related compound of this invention and adjust the therapeutic regimen accordingly. A modulator of cyclopentenone and cyclopentenone related compound biological activity can be evaluated by a variety of means including the use of a responsive reporter gene (i.e. PDGF, BMP-2, and BMP-6), assay of cyclopentenone levels, and analysis of bone growth and repair. For example, an “effective amount” for therapeutic uses is the amount of the composition comprising an a cyclopentenone compound or cyclopentenone related compound required to provide a clinically significant event, such as increase in healing rates in fracture repair; reversal of bone loss in osteoporosis; reversal of cartilage defects or disorders; prevention or delay of onset of osteoporosis; stimulation and/or augmentation of bone formation in fracture non-union and distraction osteogenesis; increase and/or acceleration of bone growth into prosthetic devices; and repair of dental defects. Such effective amounts will be determined using routine optimization techniques and are dependent on the particular condition to be treated, the condition of the patient, the route of administration, the formulation, and the judgment of the practitioner and other factors evident to those skilled in the art.

The dosage required for the disclosed compounds (for example, in osteoporosis where an increase in bone formation is desired) is manifested as a statistically significant difference in bone mass between treatment and control groups. This difference in bone mass can be seen, for example, as a 5-20% or more increase in bone mass in the treatment group. Other measurements of clinically significant increases in healing can include, for example, tests for breaking strength and tension, breaking strength and torsion, 4-point bending, increased connectivity in bone biopsies, and other biomechanical tests well known to those skilled in the art. General guidance for treatment regimens is obtained from experiments carried out in animal models of the disease of interest.

Additional dose determination methods have been described in the art. See, for example, those described in U.S. Pat. Nos. 5,326,902 and 5,234,933, and PCT Publication WO 93/25521.

### III. F. Bioassays

The effect and potency of the disclosed cyclopentenone compounds and compositions can be evaluated using in vivo bioassays. Preferred assays are the titanium enclosure model and the titanium implant model of bone regeneration, as described in Examples 1 and 2, respectively. Additional assays to determine bone growth and regeneration are described in Examples 6-8.

### IV. Therapeutic Applications

Another aspect of the present invention is a therapeutic method comprising administering to a subject a substance that modulates biological activity of cyclopentenone and cyclopentenone related compounds. The disclosed cyclopentenone and cyclopentenone related compounds can be used at a variety of sites from a variety of bone regeneration purposes. Useful sites of action include both intraoral and extraoral sites, including but not limited to spine, cranium, and craniofacial complex.

The disclosed cyclopentenone compounds and cyclopentenone related compounds can also be used for periodontal regeneration, peri-implant regeneration of other repair or regenerative procedures that involve tissues such as gingiva, periodontal ligament, bone, dentin, cementum, cartilage, and other soft tissues. This could include soft tissue lesions, such as ulcers, traumatic, and surgical wounds. Representative uses of the disclosed compounds include: repair of bone defects and deficiencies, such as those occurring in closed, open and non-union fractures; prophylactic use in closed and open fracture reduction; promotion of bone healing in plastic surgery; stimulation of bone in growth into non-cemented prosthetic joints and dental implants: elevation of peak bone mass in pre-menopausal women, treatment of growth deficiencies; treatment of periodontal disease and defects, and other tooth repair processes; increase in bone formation during distraction osteogenesis; and treatment of other skeletal disorders, such as age-related osteoporosis, post-menopausal osteoporosis, glucocorticoid-induced osteoporosis or disuse osteoporosis and arthritis. The disclosed compounds can also be useful in repair of congenital, trauma-induced or surgical resection of bone (for instance, for cancertreatment), and in cosmetic surgery. Further, the disclosed compounds can be used for limiting or treating cartilage defects or disorders, and can be useful in wound healing or tissue repair.

Antagonists of cyclopentenone and cyclopentenone related compounds can be used to treat periodontitis and to retard the development of athersclerotic calcifications.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Titanium Enclosure Model of Bone Regeneration

### 1. a. Titanium Enclosure Preparation

Titanium enclosures were prepared by trimming and carving 0.1 mm thickness titanium plate to half cylindrical shape which had 8×1.5×2 mm in length, height, and width respectively. Both ends of the enclosures were sealed with surgical grade, self curing acrylic resin to form a closed half cylinder which is open only in the sagittal direction. The enclosures were then washed in an ultrasonic cleaning machine and autoclaved to sterilize and to create a titanium oxide surface prior to use.

### 1. b. Surgical Procedures

Twenty-eight male Wistar rats, aged 8 weeks and weighing 350-400 g were randomly separated into 4 groups that received varying doses of Δ¹²PGJ₂. Two rats were housed in each cage with free access to normal laboratory diet and water ad libitum. After 7 days of acclimatization, the animals were initially anesthetized intramuscularly with Sodium pentobarbital (Nembutal sodium solution, Abbott Laboratories, Chicago, Ill.) at a dosage of 75 mg/kg of body weight. The area of intended surgery was shaved and disinfected using 90% ethyl alcohol. After pupillary reflex was inhibited, a 3-cm incision was made through skin and muscle parallel to the long axis over the dorsal aspect of both left and right femoral bone. The periosteum was incised and removed from the surface of the bone, creating an exposed area of sufficient size to place the open face of the cylindrical enclosure directly on the bone surface. Subsequently, a 5×1.5 mm transcortical defect was made through the cortical bone into the bone marrow at the midshaft region using round and cylindrical steel burs and a low speed micromotor machine. A sufficiently sterile 0.9% saline solution was used as a coolant to avoid heat-induced necrosis of surrounding bone. The femoral bone on one side of the animal was randomly chosen for treatment with Δ¹²PGJ₂, and the contralateral side was used as a control. FIG. 1 depicts a schematic diagram of the titanium enclosure model.

Δ¹²PGJ₂ (Cayman Chemical, Ann Arbor, Mich.) was diluted in 0.9% NaCl, pH 7.2, to the final concentration of 1×10⁻³, 1×10⁻⁵, 1×10⁻⁷, and 1×10⁻⁹ M. An absorbable collagen sponge (Colla-Tec Inc. Plainsboro, N.J.) 5×1.5 mm dimensions was used as a carrier. 10 μl of each Δ¹²PGJ₂ solution were loaded into the collagen carrier and then the carrier was placed inside the half cylindrical titanium enclosure. This provided a dosage of 3.0 μg, 30 ng, 0.3 ng, and 3 pg Δ¹²PGJ₂ in each of five standardized defects. The remaining volume inside the enclosure was filled with blood collected from the defect. The enclosure was inverted, placed over the defect, and stabilized by circumosseous wiring around the femoral bone using #010 orthodontic wire. The muscle and skin were then repositioned and sutured in two layers using bioresorbable suturing material (Chromic gut; Ethicon, Johnson & Johnson, Somerville, N.J.) and stainless steel wound clips (Autoclip; Becton Dickinson & Co., Parsippany, N.J.). On the control side, 0.9% NaCl without Δ¹²PGJ₂ was loaded into the collagen matrix and implanted. The animals were monitored every day after surgery for indication of post-operative reactions.

After 10 days, the animals were sacrificed by injection of an intraperitoneal overdose of sodium pentobarbital. The thoracic cavity was exposed and transcardial perfusion with 0.1% heparinized (heparin sodium 1000 units/ml) in 0.1 M phosphate buffer, pH 7.4 was performed. 4% paraformaldehyde (Fisher Scientific Company, Fair Lawn, N.J.) in 0.1 M phosphate buffer was perfused as a fixative. Following fixation, both femoral bones were dissected, removed, and then post-fixed in the same fixative for 48 hours. The enclosures were carefully removed and the samples were then decalcified using 10% ethylenediamine tetra-acetic acid (EDTA) pH7.4 at 4° C. The decalcification endpoint was determined by radiograph (approximately 10-14 days). EDTA solution was changed every 3 days. The tissues were then rinsed under running tap water for at least 2 hours and dehydrated for embedding in paraffin wax according to a routine paraffin-embedding procedure. The embedded specimens were cut in a plane perpendicular to the long axis of the femoral bone into 5 mm thickness sections using a microtome (American Optical Corp. of Southbridge, Mass.), floated in the water bath, and mounted onto slides coated with 0.5% gelatin and 0.05% chrome alum (chromium potassium sulfate). Sections were dried overnight at 37° C. in an oven to enhance adhesiveness and then stained with heamoxylin and eosin and Masson's Trichrome.

### 1. c. Bone Regeneration Quantitation

From each sample, five sections from different areas of the defect (at 0.5-1 mm intervals) were evaluated for histomorphometric quantification. Measurements were made at 4× using a microscope and a video digital camera (JAVA video analysis software; Jandel Scientific, Corte Madera, Calif.). The image analysis program calculated the area of a region of a specified color intensity. The percent defect was measured for each section by computing the ratio of the morphometric measurements of the cross-section area of the newly generated bone divided by the total cortical defect area and multiplied by 100. Three to five sections were analyzed for each femur and the data pooled to reflect a mean value for each bone defect. Percentage bone regeneration was computed for each animal, and the values for all animals within a treatment group were combined to form a group mean. The effects of Δ¹²PGJ₂ on bone regeneration were analyzed using a paired t-test. Test-control contralateral pairing within animals was maintained as appropriate.

### 1. d. PDGF, IGF, and BMP-2-6 Immunohistochemistry

After deparaffinization with Hemodee, the slides were rehydrated through a graded series of ethanol, and incubated for 10 minutes in 0.3% hydrogen peroxide in methanol to quench endogenous peroxide activity. Sections were washed in phosphate buffer, blocked for 20 minutes with 10% normal goat serum (for PDGF and IGF staining) or 10% normal rabbit serum (for BMP-2 and BMP-6 staining), and incubated at 4° C. overnight in a 1:20 dilution of anti-human PDGF-A (Research Diagnostic Inc., Flander, N.J.), a 1:30 dilution of rabbit anti-human PDGF-BB (Research Diagnostic Inc., Flander, N.J.), or a 1:25 dilution of mouse anti-human IGF-I (Research Diagnostic Inc., Flander, N.J.). For BMP-2 and BMP-6 staining, a 1:20 dilution of goat anti-human BMP-2 and BMP-6 (Research Diagnostic Inc., Flander, N.J.) were used overnight at 4° C. and 1 hour at room temperature, respectively. After extensive washing, appropriate biotinylated secondary antibodies (Zymed Laboratories, Inc., South San Francisco, Calif.) were applied for 30 minutes. The sections were washed again and avidin-biotin peroxides complex were applied for 30 minutes. Finally, the sections were incubated in a solution containing 3-amino-9-ethylcarbazole and 0.02% hydrogen peroxide for 6 minutes. Tissue sections were counterstained with hematoxylin for 1 minute.

### 1. e. Quantitative Evaluation of PDGF, IGF, BMP-2, and BMP-6 Expression

The area and average intensity of positive immunostaining were measured using a microscope connected to a computerized video digital system (JAVA video analysis software, Jandel Scientific, Corte Madera, Calif.) at a magnification of 100×. Three to five sections were analyzed for each femur and the data from all sections were pooled to reflect a mean value for each bone defect. Animals within treatment groups were combined to form a group mean and estimate of standard error deviation. Results were expressed in percentage of positive immunostaining area per cortical defect and average intensity respectively.

### 1. f. Statistical Analysis

All results indicated as a mean value, derived from at least three sections from each defect in each group, +/− standard error of the mean (SEM). Comparison among the groups were made with a one-way analysis of variance (ANOVA) and student's t-test. The significance level was designated as p<0.05.

Two animals died during surgical anesthesia and were not replaced. All other animals rapidly recovered following surgery and remained in excellent health throughout the course of the experiment. All rats applied full weight to their hind limbs within one day after surgery. At the time of sacrifice, two animals had a presumptive infection on one of the two surgical sites and were therefore eliminated from the analysis.

### Example 2

### Titanium Implant Model of Bone Regeneration

### 2. a. Surgical Placement of Implants

Six male Wistar rats were anesthetized as described in Example 1 for the placement of 4 titanium implants in each animal. Implants were fabricated from surgical grade titanium 1.5 mm in diameter, 2.5 mm in length with machined threads, cleaned bysonication and autoclaved prior to use. Implant sites were prepared with cooling using a dental high speed hand piece. One test and one control implant site were prepared for placement of 5 μl of test or control solution, followed by implant placement. Control implants comprised vehicle only (10% carboxymethylcellulose in normal saline). For test sites, 10⁻³M or 10⁻⁵M Δ¹²PGJ₂ was applied to the site following placement of the implant. Animals were monitored for 3 or 8 weeks following implantation.

### 2. b. Histological Processing

Femur bones were collected and fixed in 4% paraformaldehyde, 0.1 M phosphate buffer. Samples were cleared through sequential washings of graded alcohol followed by xylene. Bones were embedded in methacrylate and sectioned with a low speed diamond saw and stained with Toluidine Blue. Two different quantitative measurements were derived from each implant to measure the amount of bone regenerated around each implant. These measurements represent the bone height from the endosteal surface to the most apical bone contact area of the implant and the mean width of that same bone calculated using three measurement references, including the endosteal surface (ES), ES +130 microns, and ES +260 microns.

### Example 3

### Preparation of Demineralized Bone Matrix

### 3. a. Preparation of Demineralized Bone

The method is essentially that described by Sampath and Reddi (1983) Proc Natl Acad Sci USA 80:6591-6595. Briefly, bovine diaphyseal bones (age 1-10 days) are obtained from a local slaughterhouse and used fresh. The bones are stripped of muscle and fat, cleaned of periosteum, demarrowed by pressure with cold water, dipped in cold absolute ethanol, and stored at −20° C. They are then dried and fragmented by crushing and pulverized in a large mill. Care is taken to prevent heating by using liquid nitrogen. The pulverized bone is milled to a particle size in the range of 70-850 μm, preferably 150-420 μm, and is defatted by two washes of approximately two hours duration with three volumes of chloroform and methanol (3:1). The particulate bone is then washed with one volume of absolute ethanol and dried over one volume of anhydrous ether yielding defatted bone powder. The defatted bone powder is then demineralized by four successive treatments with 10 volumes of 0.5N HCl at 4° C. for 40 min. Finally, neutralizing washes are done on the demineralized bone powder with a large volume of water.

Demineralized bone matrix thus prepared is extracted with 5 volumes of 4M guanidine-HCl, 50 mM Tris-HCl, pH 7.0 for 16 hours at 4° C. The suspension is filtered. The insoluble material is collected and used to fabricate the matrix. The material is mostly collagenous in nature. It is devoid of osteogenic or chondrogenic activity.

### 3. b. Matrix Treatments

The major component of all bone matrices is Type-I collagen. In addition to collagen, demineralized bone extracted as disclosed above includes non-collagenous proteins which can account for 5% of its mass. In a xenogenic matrix, these noncollagenous components can present themselves as potent antigens, and can constitute immunogenic and/or inhibitory components. These components also can inhibit osteogenesis in allogenic implants by interfering with the developmental cascade of bone differentiation. Treatment of the matrix particles with a collagen fibril-modifying agent extracts potentially unwanted components from the matrix, and alters the surface structure of the matrix material. Useful agents include acids, organic solvents or heated aqueous media. Various treatments are described below. A detailed physical analysis of the effect these fibril-modifying agents have on demineralized, quanidine-extracted bone collagen particles is disclosed in U.S. Pat. No. 5,171,574.

After contact with the fibril-acidifying agent, the treated matrix is washed to remove any extracted components, following a form of the procedure set forth below.

1. Suspend in TBS (Tris-buffered saline) I g/200 ml and stir at 4° C. for 2 hours; or in 6M urea, 50 mM Tris-HCl, 500 mM NaCl, pH 7.0 (UTBS) or water and stir at room temperature (RT) for 30 minutes (sufficient time to neutralize the pH);

2. Centrifuge and repeat wash step; and

3. Centrifuge, discard supernatant; water wash residue; and then lyophilize.

### 3. c. Matrix Treatments

Trifluoroacetic acid (TFA) is a strong non-oxidizing acid that is a known swelling agent for proteins, and which modifies collagen fibrils. Bovine bone residue prepared as described herein above is sieved, and particles of the appropriate size are collected. These particles are extracted with various percentages (1.0% to 100%) of trifluoroacetic acid and water (v/v) at 0C. or room temperature for 1-2 hours with constant stirring. The treated matrix is filtered, lyophilized, or washed with water/salt and then lyophilized.

Like trifluoroacetic acid, hydrogen fluoride is a strong acid and swelling agent, and also is capable of altering intraparticle surface structure. Hydrogen fluoride is also a known deglycosylating agent. As such, HF can function to increase the osteogenic activity of matrices by removing the antigenic carbohydrate content of any glycoproteins still associated with the matrix after guanidine extraction.

Bovine bone residue prepared as described above is sieved, and particles of the appropriate size are collected. The sample is dried in vacuo over P₂O₅, transferred to the reaction vessel and exposed to anhydrous hydrogen fluoride (10-20 ml/g of matrix) by distillation onto the sample at −70° C. The vessel is allowed to warm to 0° C. and the reaction mixture is stirred at this temperature for 120 minutes. After evaporation of the hydrogen fluoride in vacuo, the residue is dried thoroughly in vacuo over KOH pellets to remove any remaining traces of acid. Extent of deglycosylation can be determined from carbohydrate analysis of matrix samples taken before and after treatment with hydrogen fluoride, after washing the samples appropriately to remove non-covalently bound carbohydrates. SDS-extracted protein from HF-treated material is negative for carbohydrate as determined by ConA blotting.

The deglycosylated bone matrix is next washed twice in TBS (Tris-buffered saline) or UTBS, water-washed, and then lyophilized. Other acid treatments are envisioned in addition to HF and TFA. TFA is a currently preferred acidifying reagent in these treatments because of its volatility. However, it is understood that other, potentially less caustic acids can be used, such as acetic or formic acid.

Dichloromethane (DCM) is an organic solvent capable of denaturing proteins without affecting their primary structure. This swelling agent is a common reagent in automated peptide synthesis, and is used in washing steps to remove components.

Bovine bone residue, prepared as described herein above, is sieved, and particles of the appropriate size are incubated in 100% DCM or, preferably, 99.9% DCM/0, 1% TFA. The matrix is incubated with the swelling agent for one or two hours at 0° C. or at room temperature. Alternatively, the matrix is treated with the agent at least three times with short washes (20 minutes each) with no incubation.

Acetonitrile (ACN) is an organic solvent, capable of denaturing proteins without affecting their primary structure. It is a common reagent used in high-performance liquid chromatography, and is used to elute proteins from silica-based columns by perturbing hydrophobic interactions.

Bovine bone residue particles of the appropriate size, prepared as described herein above, are treated with 100% ACN (1.0 g/30 ml) or, preferably, 99.9% ACN/0, 1% TFA at room temperature for 1-2 hours with constant stirring. The treated matrix is then water-washed, or washed with urea buffer, or 4M NaCl and lyophilized. Alternatively, the ACN or ACN/TFA treated matrix can be lyophilized without wash.

Isopropanol is also an organic solvent capable of denaturing proteins without affecting their primary structure. It is a common reagent used to elute proteins from silica HPLC columns.

Bovine bone residue particles of the appropriate size prepared as described above are treated with 100% isopropanol (10 g/3 0 ml) or, preferably, in the presence of 0.1 % TFA, at room temperature for 1-2 hours with constant stirring. The matrix is then water-washed or washed with urea buffer or 4M NaCl before being lyophilized.

Chloroform also can be used to increase surface area of bone matrix like the reagents set forth above, either alone or acidified.

Heat treatment of matrices preferably uses a heated aqueous fibril-modifying medium such as water, to increase the matrix particle surface area and porosity. A preferred aqueous medium is an acidic aqueous medium having a pH of less than about 4.5, for example, within the range of about pH 2-pH 4 which can help to “swell” the collagen before heating. 0.1% acetic acid, ˜pH 3, is preferred. 0.1 M acetic acid also can be used.

Various amounts of delipidated, demineralized guanidine-extracted bone collagen are heated in the aqueous medium (1 g matrix/30 ml aqueous medium) under constant stirring in a water-jacketed glass flask, and maintained at a given temperature for a predetermined period of time. Preferred treatment times are about one hour, although exposure times of between about 0.5-2 hours appear acceptable. The temperature employed is held constant at a temperature within the range of about 37° C. to 65° C. The currently preferred heat treatment temperature is within the range of about 45° C. to 60° C.

After the heat treatment, the matrix is filtered, washed, lyophilized and used for implant. Where an acidic aqueous medium is used, the matrix also is preferably neutralized prior to washing and lyophilization. A currently preferred neutralization buffer is a 200 mM sodium phosphate buffer, pH 7.0. To neutralize the matrix, the matrix preferably first is allowed to cool following thermal treatment, the acidic aqueous medium (e.g., 0.1% acetic acid) then is removed and replaced with the neutralization buffer and the matrix agitated for about 30 minutes. The neutralization buffer then can be removed and the matrix washed and lyophilized.

The matrix also can be treated to remove contaminating heavy metals, such as by exposing the matrix to a metal ion chelator. For example, following thermal treatment with 0.1% acetic acid, the matrix can be neutralized in a neutralization buffer containing EDTA (sodium ethylenediaminetetraacetic acid), for example, 200 mM sodium phosphate, 5 mM EDTA, pH 7.0. 5 mM EDTA provides about a 100-fold molar excess of chelator to residual heavy metals present in the most contaminated matrix tested to date. Subsequent washing of the matrix following neutralization appears to remove the bulk of the EDTA. EDTA treatment of matrix particles reduces the residual heavy metal content of various metals (Sb, As, Be, Cd, Cr, Cu, Co, Pb, Hg, Ni, Se, Ag, Zn, TI) to less than about 1 ppm. Bioassays with EDTA-treated matrices indicated that treatment with the metal ion chelator does not inhibit bone inducing activity.

### Example 4

### Combination of Cyclopentenone Compounds with Matrix

### 4. a. Ethanol Trifluoroacetic Acid Lyophilization

In this procedure, the compound is solubilized in an ethanol triflouroacetic acid solution (47.5% EtOH/0.0, 1% TFA) and added to the matrix material. Samples are vortexed vigorously and then lyophilized.

### 4. b. Acetonitrile Trifluoroacetic Acid Lyophilization

This is a variation of the above procedure, using an acetonitrile trifluoroacetic acid (ACN/TFA) solution to solubilize the compound that then is added to the matrix material. Samples are vigorously vortexed many times and then lyophilized.

### 4. c. Ethanol Precipitation

Matrix is added to the compound dissolved in guanidine-HCl. Samples are vortexed and incubated at a low temperature (e.g., 4° C.). Samples are then further vortexed. Cold absolute ethanol (5 volumes) is added to the mixture which is then stirred and incubated, preferably for 30 minutes at −20° C. After centrifugation in a microfuge at high speed) the supernatant is discarded. The reconstituted matrix is washed twice with cold concentrated ethanol in water (85% EtOH) and then lyophilized.

### 4. d. Buffered Saline Lyophilization

Preparations of the disclosed compounds in physiological saline can also be vortexed with the matrix and lyophilized to produce osteogenically active material.

### Example 5

### Methods for Microparticle Construction

### 5. a. Emulsion Based Methods

Emulsion-based processes usually begin with the preparation of two separate phases: a first phase, which generally comprises a dispersion or solution of a disclosed compound in a solution of polymer dissolved in a first solvent, and a second phase, which generally comprises a solution of surfactant and a second solvent that is at least partially immiscible with the dispersed phase. After the first and second phases are prepared, they are combined using dynamic or static mixing to form an emulsion, in which microdroplets of the first phase are dispersed in the second, or continuous, phase. The microdroplets then are hardened to form polymeric microparticles that contain the compound. The hardening step is carried out by removal of the first solvent from the microdroplets, generally by either an extraction or evaporation process.

### 5. b. Solvent Extraction or Removal

In this method, the compound is dispersed or dissolved in a solution of the selected polymer in a volatile organic solvent like methylene chloride. This mixture is suspended by stirring in an organic oil (such as silicon oil) to form an emulsion. Several U.S. patents describe solvent removal by extraction. For example, U.S. Pat. No. 5,643,605 discloses an encapsulation process in which the emulsion is transferred to a hardening bath (i.e. extraction medium) and gently mixed for about 1 to 24 hours to extract the polymer solvent. U.S. Pat. No. 5,407,609 teaches transferring the emulsion to a volume of extraction medium that is preferably ten or more times the volume required to dissolve all of the solvent in the microdroplets, so that greater than 20-30% of the solvent is immediately removed. U.S. Pat. No. 5,654,008 similarly discloses a process in which the volume of quench liquid, or extraction medium, should be on the order of ten times the saturated volume.

Unlike solvent evaporation, this method can be used to make microspheres from polymers with high melting points and different molecular weights. Microspheres that range in diameter to between 1-300 microns can be obtained by this procedure. The external morphology of spheres produced with this technique is highly dependent on the type of polymer used.

### 5. c. Solvent Evaporation

Evaporation is another approach known in the art for solvent removal. For example, U.S. Pat. Nos. 3,891,570 and 4,384,975 teach solvent removal by evaporating an organic solvent from an emulsion, preferably under reduced pressure or vacuum. See also solvent evaporation, as described by Mathiowitz et al. (1990) J Scanning Microscopy 4:329; Beck et al. (1979) Fertil Steril 31:545; and Benita et al. (1984) J Pharm. Sci. 73:1721.

Generally, the polymer is dissolved in a volatile organic solvent, such as methylene chloride. The compound is added to the solution, and the mixture is suspended in an aqueous solution that contains a surface active agent such as poly(vinyl alcohol). The resulting emulsion is stirred until most of the organic solvent evaporated, leaving solid microspheres. The solution is loaded with antigen and suspended in vigorously stirred distilled water containing 1 % (w/v) surfactant such as poly(vinyl alcohol). The organic solvent evaporates from the polymer, and the resulting microspheres are washed with water and dried overnight in a lyophilizer. Microspheres with different sizes (1-1000 microns) and morphologies can be obtained by this method.

The foregoing methods can also be combined. For example, U.S. Pat. No. 4,389,330 describes an emulsion-based method for making drug-loaded polymeric microspheres that uses a two-step solvent removal process: evaporation followed by extraction. The evaporation step is conducted by application of heat, reduced pressure, or a combination of both, to remove between 10 and 90% of the solvent.

### 5. d. Hot-melt Encapsulation

Hot-melt encapsulation is typically used only with polymers having a low melting point, for example, polyanhydrides, and is performed, for example as described by Mathiowitz et al. (1987) Reactive Polymers 6:275. In this method, the polymer is first melted and then mixed with the solid particles of the compound that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microspheres are washed by decantation with petroleum ether to give a free-flowing powder. Microspheres with sizes between one to 1000 microns are obtained with this method. The external surfaces of spheres prepared with this technique are usually smooth and dense. This procedure is used to prepare microspheres made of polyesters and polyanhydrides. However, this method is limited to polymers with molecular weights between 1000-50,000.

### 5. e. Spray Drying

Spray drying is another common technique for making particles for drug delivery. In brief, a solution or suspension of the compound and polymer is made, then atomized under conditions removing the polymer solvent. For example, the polymer is dissolved in methylene chloride (0.04 g/mL. A known amount of the compound is suspended (insoluble compounds) or co-dissolved (soluble compounds) in the polymer solution. The solution or the dispersion is then spray-dried. Typical process parameters for a mini-spray drier (Buchi) are as follows: polymer concentration=0.04 g/mL, inlet temperature=˜24° C., outlet temperature=13-15° C., aspirator setting=15, pump setting=10 mL/minute, 15 spray flow=600 Nil/hr, and nozzle diameter=0.5 mm. Microspheres ranging between 1-10 microns are obtained with a morphology that depends on the type of polymer used. This method is primarily used for preparing microspheres having a particle size not in excess of 10.

### 5. f. Hydrogel Microparticles

Microspheres made of gel-type polymers, such as alginate, chitosan, alginate/polyethylenimide (PEI) and carboxymethyl cellulose (CMC), are produced through traditional ionic gelation techniques. The polymers are first dissolved in an aqueous solution, mixed with the compound, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microspheres are left to incubate in the bath for twenty to thirty minutes in order to allow sufficient time for gelation to occur. Microsphere particle size is controlled by using various size extruders or varying either the nitrogen gas or polymer solution flow rates.

Other hydrogel microparticle compositions comprise a reversibly gelling polymeric network. Such networks comprise a responsive polymer component capable of aggregation in response to an environmental stimulus. See PCT International Publication WO 98/06438. Preferably, the polymer network is a reversibly thermally viscosifying polymer network. The polymer network includes at least one responsive polymer component which is capable of aggregation in solution in response to an environmental stimulus and also includes at least one structural component which exhibit self-repulsive interactions under conditions of use. The responsive component is randomly bonded to the structural component. The polymer network is characterized by its ability to viscosify in response to environmental stimuli.

Preferably, the polymer network contains about 0.01-20 percent by weight of each of the response polymer and the structural polymer. Particularly preferred polymer network compositions range from a ratio of about 1:10 to about 10:1 response polymer: structural polymer. Also preferred are polymer network gel compositions which exhibit a reversible gelation at body temperature (approximately 37° C.±5° C. For particularly preferred polymers, see PCT International Publication WO 98/06438.

### 5. g. Microparticles that Release Bound Compound in Response to pH

Some polymeric materials aggregate under certain conditions to encapsulate or incorporate compound within the microparticle, then release upon exposure to a stimulus such as a change in pH or temperature. An example of microparticles that release as a function of a change in pH include the diketopiperazine particles described in U.S. Pat. No. 5,352,461 and the proteinoid formulations described in U.S. Pat. Reissue No. 35,862.

### Example 6

### Rat Model of Bone Regeneration

### 6. a. Implantation

The bioassay for bone induction as described by Sampath and Reddi (1983) can be used to monitor endochondral bone differentiation activity. This assay comprises implanting test samples in subcutaneous sites in recipient rats under ether anesthesia. Male Long-Evans rats can be used. A vertical incision (1 cm) is made under sterile conditions in the skin over the thoracic region, and a pocket is prepared by blunt dissection. Approximately 25 mg of the test sample is implanted deep into the pocket and the incision is closed with a metallic skin clip. The day of implantation is designated as day one of the experiment. Implants are removed on day 12. The heterotropic site allows for the study of bone induction without the possible ambiguities resulting from the use of orthotropic sites. Bone inducing activity is determined biochemically by the specific activity of alkaline phosphatase and calcium content of the day 12 implant. An increase in the specific activity of alkaline phosphatase indicates the onset of bone formation. Calcium content, on the other hand, is proportional to the amount of bone formed in the implant. Bone formation therefore is calculated by determining the calcium content of the implant on day 12 in rats and is expressed as “bone forming units,” where one bone forming unit represents the amount of protein that is needed for half maximal bone forming activity of the implant on day 12. Bone induction exhibited by intact demineralized rat bone matrix is considered to be the maximal bone differentiation activity for comparison purposes in this assay.

### 6. b. Cellular Events

Successful implants exhibit a controlled progression through the stages of protein-induced endochondral bone development, including: (1) transient infiltration by polymorphonuclear leukocytes on day one; (2) mesenchymal cell migration and proliferation on days two and three; (3) chondrocyte appearance on days five and six; (4) cartilage matrix formation on day seven; (5) cartilage calcification on day eight; (6) vascular invasion, appearance of osteoblasts, and formation of new bone on days nine and ten; (7) appearance of osteoclasts, bone remodeling and dissolution of the implanted matrix on days twelve to eighteen; and (8) hematopoietic bone marrow differentiation in the ossicles on day twenty-one.

### 6. c. Histological Evaluation

Histological sectioning and staining is preferred to determine the extent of osteogenesis in the implants. Implants are fixed in Bouins Solution, embedded in paraffin, and cut into 6-8 μm sections. Staining with toluidine blue or hemotoxylin/eosin demonstrates clearly the ultimate development of endochondral bone. Twelve day implants are usually sufficient to determine whether the implants contain newly induced bone.

### 6. d. Biological Markers

Alkaline phosphatase activity can be used as a marker for osteogenesis. The enzyme activity can be determined spectrophotometrically after homogenization of the implant. The activity peaks at 9-10 days in vivo and thereafter slowly declines. Implants showing no bone development by histology have little or no alkaline phosphatase activity under these assay conditions. The assay is useful for quantification and obtaining an estimate of bone formation quickly after the implants are removed from the rat. Alternatively, the amount of bone formation can be determined by measuring the calcium content of the implant.

### Example 7

### Feline Model of Bone Regeneration

The feline model can be used as a large animal efficacy model for the testing of the disclosed compounds, and to characterize repair of massive bone defects and simulated, fracture non-union encountered frequently in the practice of orthopedic surgery. The model is designed to evaluate whether implants of the disclosed compounds with a carrier can enhance the regeneration of bone following injury and major reconstructive surgery by use of this large mammal model. The first step comprises the surgical preparation of a femoral osteotomy defect which, without further intervention, would consistently progress to non-union of the simulated fracture defect. The effects of implants of the disclosed compounds (in implantable devices) into the created bone defects are evaluated as described herein below.

### 7. a. Surgical Procedure

Adult cats undergo unilateral preparation of a 1 cm bone defect in a femur through a lateral surgical approach. The femur is immediately internally fixed by lateral placement of an 8-hole plate to preserve the exact dimensions of the defect. Next, an implant containing a compound as disclosed is implanted in the surgically created cat femoral defects.

All animals are allowed to ambulate ad libitum within their cages post operatively. All cats are injected with tetracycline (25 mg/kg subcutaneously (SQ) each week for four weeks) for bone labeling. The animals are sacrificed four months after femoral osteotomy.

### 7. b. Radiomorphometrics

In vivo radiomorphometic studies are carried out at 0, 4, 8, 12 and 16 weeks following operation by taking a standardized X-ray of the lightly anesthetized animal positioned in a cushioned X-ray jig designed to consistently produce a true anterior-posterior view of the femur and the osteotomy site. All X-rays are taken in exactly the same fashion and in exactly the same position on each animal. Bone repair is calculated as a function of mineralization by means of random point analysis. A final specimen radiographic study of the excised bone is taken in two planes after sacrifice.

### 7. c. Biomechanics

Excised test and normal femurs are immediately studied by bone densitometry, or wrapped in two layers of saline-soaked towels, placed into sealed plastic bags, and stored at −20° C. until further study. Bone repair strength, load to failure, and work to failure are tested by loading to failure on a specially designed steel 4-point bending jig attached to a suitable testing machine from Instron Corporation of Canton, Mass. to quantitate bone strength, stiffness, energy absorbed and deformation to failure, and other such measurements determined by using an Instron machine.

The study of test femurs and normal femurs yield the bone strength (load) in pounds and work to failure in joules.

### 7. d. Histomorphometry/Histology

Following biomechanical testing the bones are immediately sliced into two longitudinal sections at the defect site, weighed, and the volume measured.

One half is fixed for standard calcified bone histomorphometrics with fluorescent stain incorporation evaluation, and the other half is fixed for decalcified hemotoxylin/eosin stain histology preparation.

### 7. e. Biochemistry

Selected specimens from the bone repair site are homogenized in cold 0.1 5 M NaCl, 3 mM NaHCO₃, pH 9.0 by a SPEX Centriprep 6750™ freezer mill from SPEX Centriprep pf Mituchen, N.J. The alkaline phosphatase activity of the supernatant and total calcium content of the acid soluble fraction of sediment are then determined.

### Example 8

### Rabbit Model of Bone Regeneration

Mature (less than 10 lbs) New Zealand White rabbits with epiphyseal closure documented by X-ray are used as a model in which there is minimal or no bone growth in the control animals, so that when bone induction is tested, only a strongly inductive substance will yield a positive result. Defects of 1.5 cm are created in the rabbits, with implantation of a disclosed compound.

In another assay, the marrow cavity of the 1.5 cm ulnar defect can be packed with activated disclosed compound rabbit bone powder and the bones allografted in an intercalary fashion. Healing of the lesions is then monitored and compared to control.

### REFERENCES

The publications and other materials listed below and/or set forth in the text above to illuminate the background of the invention, and in particular cases, to provide additional details respecting the practice, are fully incorporated herein by reference.

Aspinall (1970) “Polysaccharides” Pergamon Press, Oxford, UK.

Bangham et al. (1965) J Mol Biol 23:238-252.

Beck et al. (1979) Fertil Steril 31:545.

Beck et al. (1999) Am Heart J 138(5 PT 2):S528-533.

Benita (ed) (1996) “Microencapsulation: Methods and Industrial Applications” Marcel Dekker, Inc., New York.

Benita et al. (1984) J Pharm. Sci. 73:1721.

Bilezlkian et al. (eds) “Principles of Bone Biology” pp. 15-28 Academic Press, San Diego, Calif., USA.

Caplan (1991) J Orthop Res 9:641-650.

Caner et al. (1986) Biochim Biophys Acta 858:161-168.

Dover et al. (1994) Histochemistry 102(5):383-387.

Doubrow (ed) (1992) “Microcapsules and Nanoparticles in Medicine and Pharmacy” CRC Press, Boca Raton.

Feyen et al. (1984) Prostaglandins 28(6):769-778.

Horner et al. (1996) Bone 19(4):353-362.

Hotz et al. (1994) Oral & Maxillofacial Surg 23:413-417.

Howell et al. (1997) J Periodontol 68(12): 1186-1193.

Huang et al. (2000) Pac Symp Biocomput 230-41.

Hughes et al. (1992) Bone & Mineral 19(1):63-74.

Hutchens and Yip (1993) Rapid Commun Mass Spectrom 7:576-580.

Jee (1992) Bone 13(2):153-159.

Jee and Ma (1997) Bone 21(4):297-304.

Kawaguchi et al. (1996) Clin Orthopaed Rel Res 313:36-46.

Kim et al. (1983) Biochim Biophys Acta 728:339-348.

Klein-Nulend et al. (1997) Bone & Min Res 12(1):45-51.

Lesernian et al. (1980) Nature 288:602-604.

Liedberg et al. (1983) Sensors Actuators 4:299-304.

Malmquist (1993) Nature 361:186-187.

Madge et al. (1972) Phys Rev Lett 29:705-708.

Maiti et al. (1997) Proc Natl Acad Sci USA, 94:11753-11757.

Mathiowitz et al. (1987) Reactive Polymers 6:275.

Mathiowitz et al. (1990) J Scanning Microscopy 4:329.

Min et al. (1998) J Bone Miner Res 13:1066-1075.

Nemoto (1997) Prostaglandins 54:713-725.

Offenbacher et al. (1999) J Periodontal Res 34(7):346-352.

Olson et al. (1979) Biochim Biophys Acta 557:9-23.

Ono et al. (1998) J Endocrinol 158:R1-5.

Raisz (1999) Osteoarthritis and Cartilage 7:419-421.

Sampath and Reddi (1983) Proc Natl Acad Sci USA 80:6591-6595.

Sampath et al. (1987) Proc Natl Acad Sci USA 84:109-113.

Saqi et al. (1999) Bioinformatics 15:521-522.

Szoka et al. (1978) Proc Natl Acad Sci USA 75:4194-4198.

Tai et al. (1997) Endocrinol 138:2372-2379.

Tencer et al. (1989) J Biomed Mat Res 23:571-589.

U.S. Pat. No. 3,891,570

U.S. Pat. No. 4,384,975

U.S. Pat. No. 4,389,330

U.S. Pat. No. 4,761,471

U.S. Pat. No. 5,171,574

U.S. Pat. No. 5,234,933

U.S. Pat. No. 5,326,902

U.S. Pat. No. 5,352,461

U.S. Pat. No. 5,407,609

U.S. Pat. No. 5,643,605

U.S. Pat. No. 5,654,008

U.S. Pat. No. 5,834,228

U.S. Pat. No. 5,872,011

U.S. Pat. Reissue No. 35,862

WO 90/11366

WO 93/25521

WO 98/06438

Woodiel (1996) J Bone Miner Res 11:1249-1255.

Worrall et al. (1998) Anal Biochem 70:750-756.

It will be understood that various details of the invention can be changed without departing from the scope of the invention. Furthermore, the foregoing description is for the purpose of illustration only, and not for the purpose of limitation—the invention being defined by the claims.

What is claimed is:

1. A method of stimulating bone growth, the method comprising:
(a) preparing a composition comprising a cyclopentenone compound, a cyclopentenone related compound, a cyclopentenone modulator, or combinations thereof, and a carrier; and
(b) administering the composition of (a) to a subject, whereby bone growth is stimulated.

2. The method of claim 1, wherein the cyclopentenone compound is selected from the group consisting of Δ¹²-13,14-dihydro-9-deoxy-Δ⁹-prostaglandin D₂, 9-hydroxy-prostaglandin D₂, prostaglandin D₂, derivatives thereof, salt forms thereof, and combinations thereof.

3. The method of claim 1, wherein the cyclopentenone compound or cyclopentenone related compound is of the formula 2-R¹, 1-R², cyclopent 3-en 1-one or 4-hydroxy, 3-R¹, 2-R², cyclopentan-1-one, wherein R¹ and R² are each independently aliphatic chains.

4. The method of claim 3, wherein the R¹ and R² are each independently substituted or unsubstituted C₁-C₁₀ straight chain or branched alkyl, substituted or unsubstituted C₂-C₁₀ straight chain or branched alkenyl, or substituted or unsubstituted C₂-C₁₀ straight chain or branched alkynyl; and wherein the substituents are each independently halogen, cyano, amino, carboxy, ester, ether, carboxamide, hydroxy, or mercapto.

5. The method of claim 1, wherein the cyclopentenone related compounds comprise:
(a) compounds that induce or enhance cyclopentenone synthesis by a cell; or
(b) compounds that are cyclopentenone agonists.

6. The method of claim 1, wherein the pharmaceutically acceptable carrier is selected from the group consisting of a membrane, a film, a matrix, a scaffold, an implantable device, a biodegradable carrier, a slow release carrier, a controlled release carrier, a liposome, and a microparticle.

7. The method of claim 6, wherein the implantable device is a collagen sponge.

8. The method of claim 6, wherein the implantable device is a titanium support.

9. The method of claim 1, wherein the composition is administered to the subject at a site of bone injury or disease.

10. The method of claim 9, wherein the site is an intraoral site.

11. The method of claim 10, further comprising implantation of a tooth or a tooth implant.

12. The method of claim 1, wherein the bone growth comprises an increase in bone volume.

13. The method of claim 1, wherein the bone growth comprises elevated expression of PDGF, BMP-2, BMP-6, and combinations thereof.

14. A therapeutic composition for bone repair comprising:
(a) a cyclopentenone, cyclopentenone related compound, or cyclopentenone regulator; and
(b) an implantable pharmaceutically acceptable carrier.

15. The composition of claim 14, wherein the cyclopentenone compound is selected from the group consisting of Δ¹²-13,14-dihydro-9-deoxy-Δ⁹-prostaglandin D₂, 9-hydroxy-prostaglandin D₂, prostaglandin D₂, derivatives thereof, salt forms thereof, and combinations thereof.

16. The composition of claim 14, wherein the cyclopentenone compound or cyclopentenone related compound is of the formula 2-R¹, 1-R², cyclopent 3-en 1-one or 4-hydroxy, 3-R¹, 2-R², cyclopentan-1-one, wherein R¹ and R₂are each independently aliphatic chains.

17. The composition of claim 16, wherein the R¹ and R² are each independently substituted or unsubstituted C₁-C₁₀ straight chain or branched alkyl, substituted or unsubstituted C₂-C₁₀ straight chain or branched alkenyl, or substituted or unsubstituted C₂-C₁₀ straight chain or branched alkynyl; and wherein the substituents are each independently halogen, cyano, amino, carboxy, ester, ether, carboxamide, hydroxy, or mercapto.

18. The composition of claim 14, wherein the cyclopentenone related compounds comprise:
(a) compounds that induce or enhance cyclopentenone synthesis by a cell; or
(b) compounds that are cyclopentenone agonists.

19. The composition of claim 14, wherein the pharmaceutically acceptable carrier is selected from the group consisting of a membrane, a film, a matrix, a scaffold, an implantable device, a biodegradable carrier, a slow release carrier, a controlled release carrier, a liposome, and a microparticle.

20. The composition of claim 19, wherein the implantable device is a collagen sponge.

21. The composition of claim 19, wherein the implantable device is a titanium support.

22. A method for identifying a cyclopentenone modulator, the method comprising:
(a) providing a cyclopentenone or cyclopentenone related compound;
(b) exposing the cyclopentenone or cyclopentenone related compound to a plurality of candidate modulators; and
(c) selecting a candidate regulator that demonstrates specific binding to the cyclopentenone or cyclopentenone related compounds, whereby a cyclopentenone modulator is identified.

23. The method of claim 22, wherein the cyclopentenone modulator is selected from the group consisting of a protein, a peptide, an antibody, a chemical compound, and a nucleic acid.

24. The method of claim 22, wherein the cyclopentenone compound is selected from the group consisting of Δ¹²-13,14-dihydro-9-deoxy-Δ⁹-prostaglandin D₂, 9-hydroxy-prostaglandin D₂, prostaglandin D₂, derivatives thereof, salt forms thereof, and combinations thereof.

25. The method of claim 22, wherein the cyclopentenone compound or cyclopentenone related compound is of the formula 2-R¹, 1-R², cyclopent 3-en 1-one or 4-hydroxy, 3-R¹, 2-R², cyclopentan-1-one, wherein R¹ and R² are each independently aliphatic chains.

26. The method of claim 25, wherein the R¹ and R² are each independently substituted or unsubstituted C₁-C₁₀ straight chain or branched alkyl, substituted or unsubstituted C₂-C₁₀ straight chain or branched alkenyl, or substituted or unsubstituted C₂-C₁₀ straight chain or branched alkynyl; and wherein the substituents are each independently halogen, cyano, amino, carboxy, ester, ether, carboxamide, hydroxy, or mercapto.
